(19) United States
(12) Patent Application Publication
(10) Pub. No.: US 2024/0309079 A1
(43) Pub. Date: Sep. 19, 2024

(54) COMBINATION TREATMENT OF CHRONIC MYELOMONOCYTIC LEUKEMIA IN PATIENTS WITH RAS PATHWAY MUTATIONS

(71) Applicants: TARAN THERAPEUTICS, INC., Toms River, NJ (US); THE UNIVERSITY OF ADELAIDE, Adelaide (AU)

(72) Inventors: Cameron DURRANT, Bella Collina, FL (US); Daniel THOMAS, Adelaide (AU); Dale CHAPPELL, Dolores, CO (US)

(73) Assignees: TARAN THERAPEUTICS, INC., Toms River, NJ (US); THE UNIVERSITY OF ADELAIDE, Adelaide, SA (AU)

(21) Appl. No.: 18/604,466

(22) Filed: Mar. 13, 2024

Related U.S. Application Data
(60) Provisional application No. 63/595,285, filed on Nov. 1, 2023.
(60) Provisional application No. 63/452,147, filed on Mar. 14, 2023.

(51) Int. Cl. C07K 16/24; A61K 31/706; A61K 31/7072; A61K 39/00; A61K 39/395; A61P 35/02

(52) U.S. Cl. CPC C07K 16/243; A61K 31/706; A61K 31/7072; A61K 39/3955; A61P 35/02; A61K 2039/505; A61K 2039/54; A61K 2039/545; C07K 2317/21

(57) ABSTRACT

Provided herein are methods for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody. The subject may have a RAS pathway mutation or a RAS pathway mutation and at least one TET2 mutation identified in the tumor cells. The methods further comprise administering a therapeutically effective amount of a hypomethylating agent. Also provided herein are methods for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody lenzilumab and a therapeutically effective amount of a hypomethylating agent. The subject may have a RAS pathway mutation or a RAS pathway mutation and at least one TET2 mutation identified in the tumor cells. A therapeutically effective amount of a hypomethylating agent is further administered according to the provided methods.

### FIELD OF THE INVENTION

The invention relates to methods for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody. In some aspects, the methods for treating CMML further comprise administering to the subject a therapeutically effective amount of an anti-hGM-CSF antibody and a therapeutically effective amount of a hypomethylating agent. The invention further relates to methods for treating a subject having CMML, the methods comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody and a therapeutically effective amount of a hypomethylating agent.

### BACKGROUND OF THE INVENTION

Chronic myelomonocytic leukemia (CMML) is a rare, aggressive cancer for which no targeted therapy exists. Standard of care (SOC) includes hypomethylating agents such as azacitidine (A) and decitabine (D), with complete and partial response (CR and PR) rates ranging between 10-17%. The pro-inflammatory cytokine granulocyte-macrophage colony-stimulating factor (GM-CSF) can play a role in stimulating leukemic cell proliferation.

Accordingly, there remains a critical need for developing improved compositions and methods for treatment of CMML, for achieving complete response (CR) plus partial response (PR) that is higher than the CR and PR rate demonstrated with sole administration of a hypomethylating agent, and for improving survival and progression-free survival after treatment compared to survival and progression-free survival after administration of a hypomethylating agent alone.

### SUMMARY OF THE INVENTION

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody.

In another aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody and a therapeutically effective amount of a hypomethylating agent.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1B show the results of Phase II DNA methyltransferase inhibitor trials (FIG. 1A) and the morphologic criteria for diagnosis of CMML including identification and quantitation of blast cells and blast-equivalent cells, including promonocytes (FIG. 1B).

FIGS. 2A-2C show the baseline characteristics of the lenzilumab cohort in the studies of Example 1: age (FIG. 2A), sex at birth (FIG. 2B), and spleen size in cm (FIG. 2C).

FIGS. 3A-3B show the baseline characteristics of the lenzilumab cohort in the studies of Example 1: cytogenetics (FIG. 3A) and bone marrow (BM) Blast % (FIG. 3B).

FIGS. 4A-4B show the baseline characteristics of the lenzilumab cohort in the studies of Example 1: whether a subject is transfusion dependent (FIG. 4A) and the amount of hemoglobin (g/L) in a subject's blood (FIG. 4B).

FIGS. 5A-5B show the baseline characteristics of the lenzilumab cohort in the studies of Example 1: white blood cell count (FIG. 5A) and monocytes (FIG. 5B).

FIG. 6 shows in tabular format the baseline characteristics of the lenzilumab cohort in the studies of Example 1: the gene mutation profile, including NRAS, KRAS, and/or CBL mutations and TET2 mutations, of 11 patients.

FIG. 7 shows in a pie chart format the genes mutated in the lenzilumab cohort. Cbl mutations are frequent, as are TET2 mutations.

FIGS. 8A-8B show the baseline characteristics of the lenzilumab cohort in the studies of Example 1: CPSS-MOL Score (FIG. 8A) and the constitutional symptoms of fatigue, weight loss and night sweats(Fig. 8B). CPSS-MOL Score is a CMML-specific prognostic scoring system (CPSS) that incorporates molecular genetic data resulting in a 4-level integrated clinical/pathological/genetic risk stratification tool.

FIG. 9 shows excellent response rates in lenzilumab-azacitidine (Lenz-Aza) combination therapy: 83% CR rate, 100% ORR (CR+Clinical Benefit). Clinical benefit includes platelet response.

FIG. 10 shows in a tabular format the baseline characteristics of the lenzilumab cohort in the studies of Example 1: CPSS-MOL Score; blast %, Hb/WCC/Platelet (Plt) x10⁹/L and monocyte count; the response after 3 cycles of combination therapy (lenz+aza) of blast %, Hb/WCC/Platelet (Plt)×10⁹/L and monocyte count; the best response (complete remission or platelet response; and the Myeloproliferative Neoplasm (MPN) total symptom score.

FIG. 11 shows the Savona criteria for myelodysplastic/myeloproliferative neoplasms (MPN/MDS) overlap syndromes in tabular format. Savona et al. (2015) proposed response assessment guidelines to harmonize future clinical trials with the principal objective of establishing suitable treatment algorithms based on the recommendations of an international panel comprising laboratory and clinical experts in MDS/MPN involving three independent academic MDS/MPN workshops (two in 2013 and one in 2014).

FIG. 12 shows the superior early response data of Lenz+Aza combination therapy for CMML compared to published literature for CMML treatment with DNA methyltransferase inhibitors (DNMTi) alone.

FIG. 13 shows high CR response rate (%) obtained from Lenz+Aza combination therapy in high risk CMML patients.

FIG. 14 shows the robust improvement in quality of life based on the MPN symptoms assessment form: total symptom score compared to baseline scores. The best responding symptoms were fatigue, weight loss, poor concentration, and inactivity.

FIG. 15 shows in tabular format certain serious adverse events that occurred in the lenzilumab cohort in the Lenz+Aza combination therapy for CMML.

FIG. 16 shows in tabular format the grade 3 & 4 adverse events of the lenzilumab cohort in the Lenz+Aza combination therapy for CMML.

FIG. 17 shows in tabular format the patient characteristics from a prospective phase II trial of azacytidine therapy in CMML patients (Drummond et al, Leukemia 2014). 32 patients with CMML-1 (70%) or CMML-2 (27%) treated with azacitidine 75 mg/m2 were of a median age of 70; had genetic mutations (TET2 63%, ASXLI 38%, EZH2 4%, CBL 8%, NRAS 5%); and median WCC 15.9 x 109/L.

FIGS. 18A-18B show lenzilumab in addition to Azacitidine improves complete response rates in chronic myelomonocytic leukemia. FIG. 18A shows a Swimmer plot showing ongoing treatment of patients with RAS-pathway mutations on LENZ/AZA arm of the study. Black arrow indicates patient has not withdrawn/progressed. FIG. 18B shows column graphs showing decreased bone marrow blast % with LENZ+AZA treatment assessed after 3, 6 and 2 months of combination treatment. P values reflect unpaired students t-test for groups as shown.

FIGS. 19A-19B show decreased size of RAS-pathway mutant clones after lenzilumab +Azacitidine. FIG. 19A shows a Circos plot showing all variant somatic mutations identified at screening in CMML patients. FIG. 19B shows column graphs showing decrease of KRAS and CBL VAF (%) in 4 individuals after LENZ+AZA treatment.

FIGS. 20A-20B show improvement in inflammatory parameters in CMML patients treated with lenzilumab and Azacitidine. FIG. 20A shows column graphs showing decrease in c-reactive protein and bone marrow GM-CSF after 3, 6 and 12 month cycles. FIG. 20B shows a heatmap showing hierarchical clustering of cytokine levels measured from CMML patient bone marrow interstitial fluid obtained at screening (screen) compared to age-matched healthy controls (HC). Two discrete clusters of CMML are apparent, INNATE-1 and INNATE-2. Patients with carly response shown in orange blocks (sensitive).

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment for CMML, including prophylactic treatment, with the pharmaceutical compositions according to the present invention, i.e., the anti-hGM-CSF antibodies and the hypomethylating agents, respectively, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

CMML is a rare disease, which occurs in about four of every one million people in the U.S. each year; about 1,100 new cases of CMML are diagnosed annually. About nine out of ten cases are found in people 60 years of age and older. CMML occurs more often in men than in women and is very rare in young people.

CMML is a clonal stem cell disorder with features of both myelodysplasia and myeloproliferative disease; monocytes in the bone marrow begin to grow out of control, filling the bone marrow and preventing other blood cells from growing.

It is thought that Clonal Hematopoiesis of Indeterminate Potential (CHIP)/clonal hematopoiesis (CH) may give rise to CMML. While the exact cause of CMML is unknown, there are some known risk factors that increase the chances of getting CMML, including older age (60 or older), being male, being exposed to certain chemicals at work or in the environment, being exposed to radiation and past treatment with certain anticancer drugs.

Diagnosis of CMML includes persistent monocytosis>1×10⁹/L with an acquired clonal abnormality (excluding PDGFR/FGFRI/BCRABL/JAK2, ET/PRV/MF) and >94% CD14⁺CD16⁻ classic monocytes are pathonognomic of CMML. 60% of CMML patients have aTET2 mutation and 40% have a NRAS/KRASICBL mutation. The median overall survival after CMML diagnosis is 30 months.

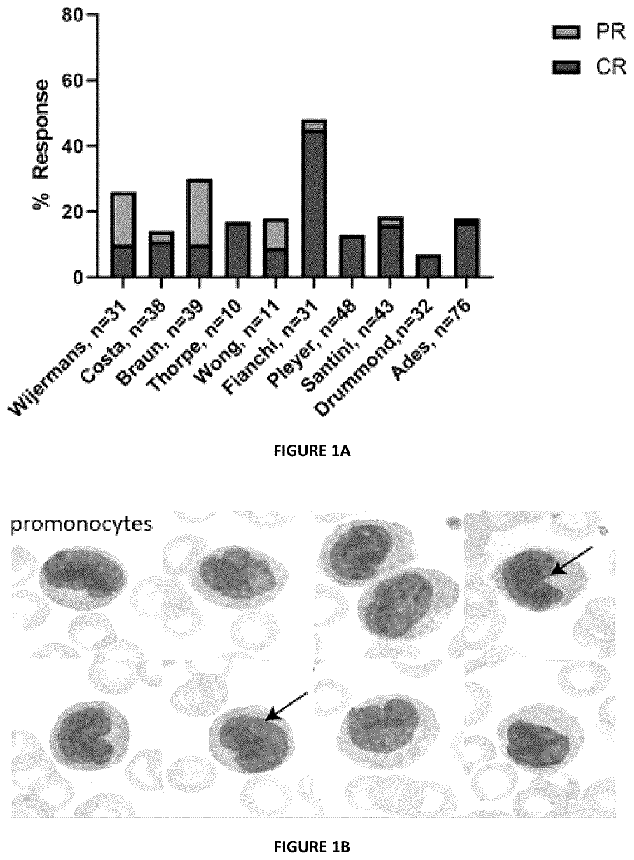

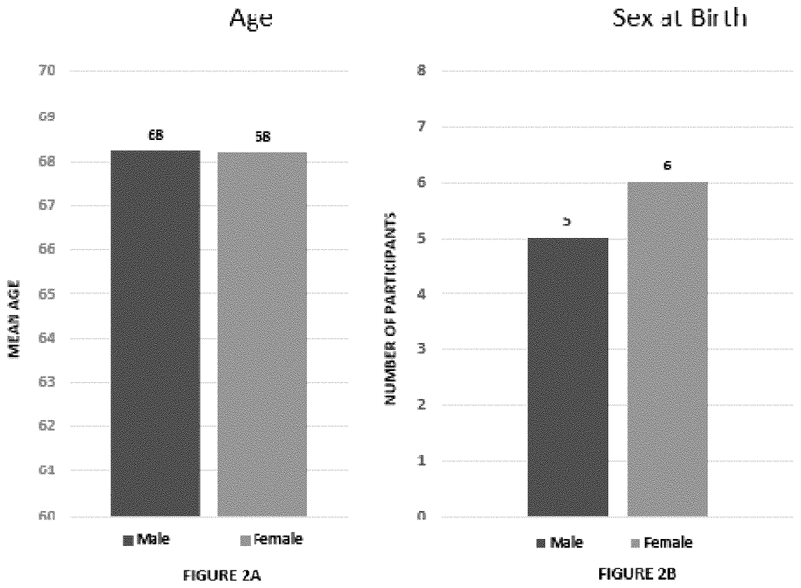

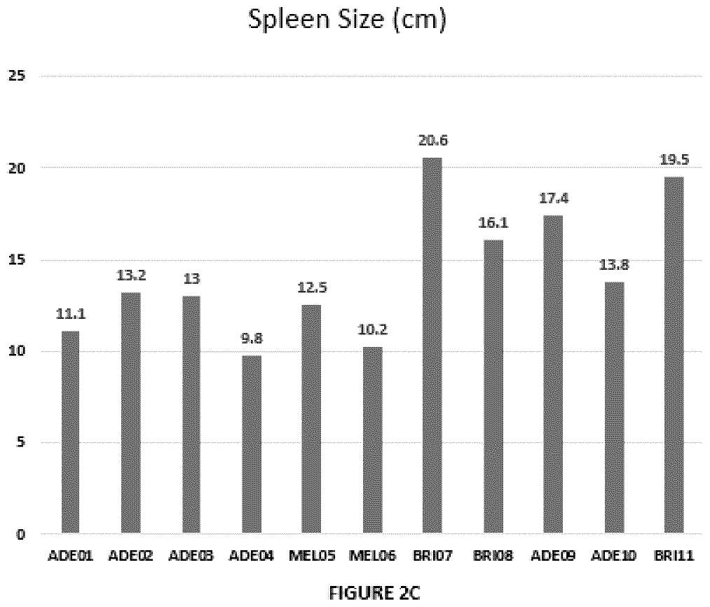

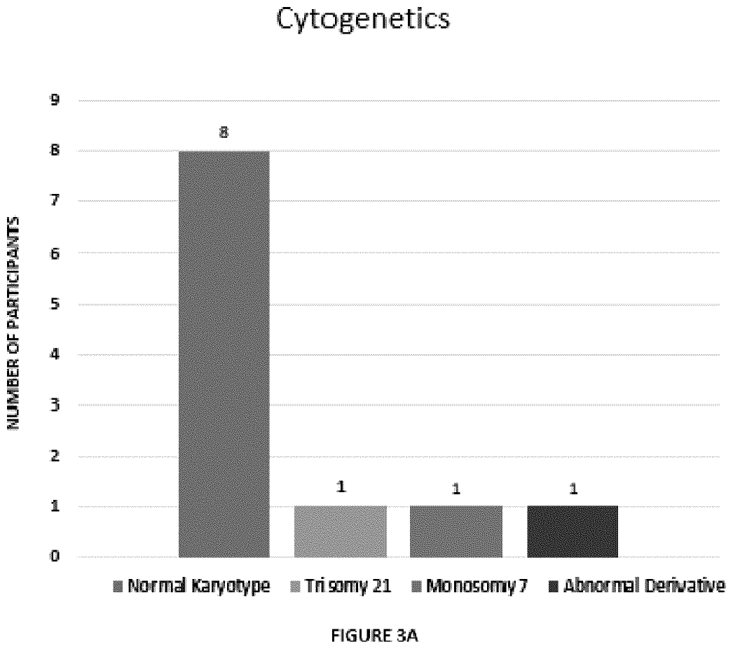

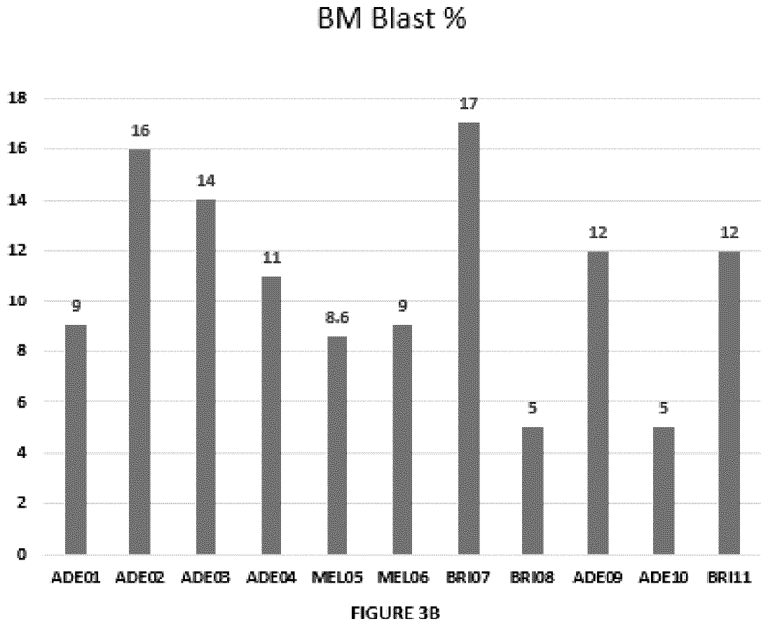

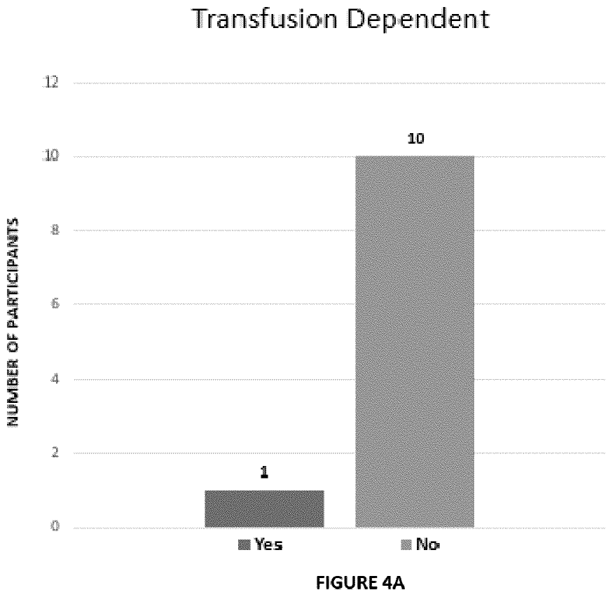

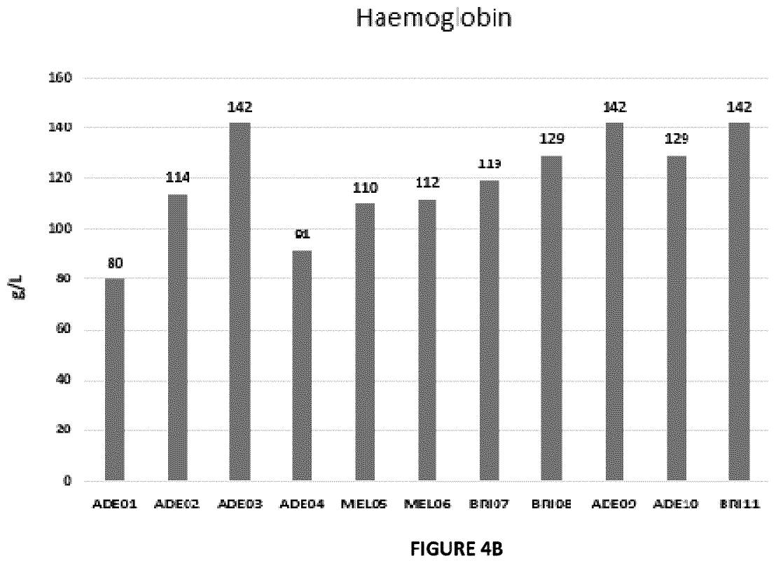

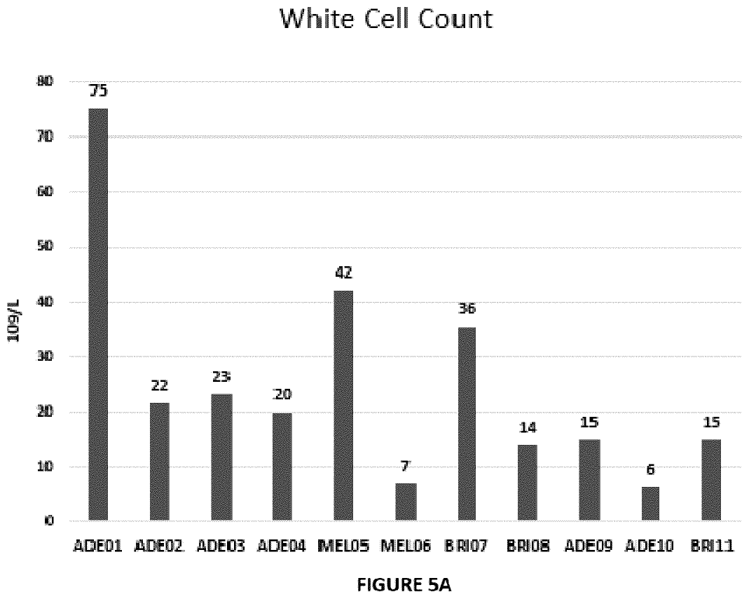

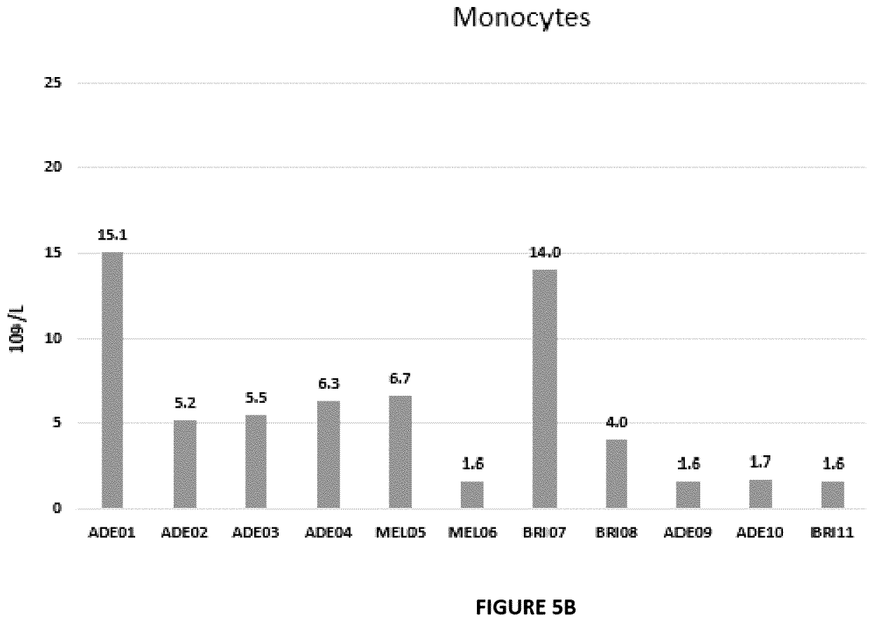

At present, azacitidine and decitabine, the only approved treatments for a select population of CMML patients leads to response rates (CR+PR) of 10-17% with limited survival benefit. FIG. 1A shows a combination of ten studies upon which the CR+PR rate for azacitidine alone in CMML of 10-17% is based.

To improve survival and progression-free survival of CMML patients after treatment compared to survival and progression-free survival after administration of a hypomethylating agent alone, which is the current SOC, a clinical trial for the treatment of CMML with combination therapy of anti-hGM-CSF antibody lenzilumab and azacitidine was commenced, as described in Example 1.

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody. In an embodiment, the anti-hGM-CSF antibody is lenzilumab. In some embodiments, the anti-hGM-CSF antibody is selected from the group consisting of Namilumab, Otilimab, Gimsilumab, and TJM2 (TJ003234). In an embodiment, the method further comprises administering a therapeutically effective amount of a hypomethylating agent for five to seven days starting on day one of administration of the anti-hGM-CSF antibody. In certain embodiments, the hypomethylating agent is selected from the group consisting of azacytidine, decitabine, and a combination of decitabine and cedazuridine (INQOVI®). In an embodiment of the herein provided methods further comprising administering a therapeutically effective amount of a hypomethylating agent, the anti-hGM-CSF antibody is lenzilumab. In certain embodiments of said methods, the anti-hGM-CSF antibody is selected from the group consisting of Namilumab, Otilimab, Gimsilumab, and TJM2 (TJ003234). In some embodiments, the anti-hGM-CSF antibody, lenzilumab is administered on day one and day 15 of cycle 1 and on day one only for all subsequent cycles. In certain embodiments, the all subsequent cycles consist of a total of 24 cycles, and cach cycle consists of 28 days. In various embodiments, the anti-hGM-CSF antibody, Namilumab, Otilimab, Gimsilumab, or TJM2 (TJ003234) is administered on day one and day 15 of cycle 1 and on day one only for all subsequent cycles. In some embodiments, the all subsequent cycles consist of a total of 24 cycles, and cach cycle consists of 28 days. In a particular embodiment, the subject has a RAS pathway mutation and a TET2 mutation. In some embodiments, the subject has a RAS pathway mutation and two TET2 mutation variants. In certain embodiments of the herein provided methods, the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hour(s) IV infusion. In a particular embodiment, the anti-hGM-CSF antibody lenzilumab is administered at a dose of 552 mg over a 1 hour IV infusion. In an embodiment, the hypomethylating agent is administered subcutaneously at a dose of 75 mg/m2. In certain embodiments, the hypomethylating agent is selected from the group consisting of azacytidine, decitabine, and a combination of decitabine and cedazuridine (INQOVI®). In a particular embodiment, the hypomethylating agent is azacitidine. In certain embodiments, the combination therapy, i.c., the administration of the anti-hGM-CSF antibody and the hypomethylating agent, demonstrates, i.c., achieves a complete response (CR) plus partial response (PR) of 50-90% compared to a CR+PR rate of 10-17% achieved with sole administration of a hypomethylating agent, c.g., azacytidine, decitabine, or a combination of decitabine and cedazuridine (INQOVI®). In a particular embodiment, the anti-hGM-CSF antibody is lenzilumab. In some embodiments of said combination therapy methods, the anti-hGM-CSF antibody is selected from the group consisting of Namilumab, Otilimab, Gimsilumab, and TJM2 (TJ003234). In an embodiment, the response to said combination therapy, i.c., the administration of the anti-hGM-CSF antibody and the hypomethylating agent is a complete response or a partial response during 6 first cycles. In a particular embodiment, the CR or PR result in improved survival and progression-free survival at two years after treatment compared to survival and progression-free survival at two years after administration of a hypomethylating agent alone. In certain embodiments, the administration of the combination therapy of the anti-hGM-CSF antibody and the hypomethylating agent demonstrates, that is, achieves, clinical benefit at any point during 24 cycles. In some embodiments of the herein provided methods, the clinical benefit comprises impact on physical and functional capacity of the subject; social well-being of the subject, hematological and non-hematologic safety and combinations thereof. In an embodiment of the provided methods, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In some embodiments, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In certain embodiments, the clinical benefit comprising impact on the physical capacity of the subject comprises a reduction in splenomegaly. In some embodiments, the hematological safety comprises a decrease in a variant allele frequency (VAF) of at least one identified RAS-pathway mutation, wherein the RAS-pathway mutation is KRAs and/or CBL. In a particular embodiment, the provided methods further comprise treating the subject with an allogencic transplant.

In another aspect, provided herein is a method for treating a subject having chronic myclomonocytic leukemia (CMML), the method comprising: (a) identifying a RAS pathway mutation in tumor cells of the subject, wherein the RAS pathway mutation is a NRAS, KRAS, and/or CBL mutation; and (b) administering to the subject identified in step (a) a therapeutically effective amount of an anti-hGM-CSF antibody and a therapeutically effective amount of a hypomethylating agent. In an embodiment, the anti-hGM-CSF antibody is lenzilumab. In some embodiments, the anti-hGM-CSF antibody is selected from the group consisting of Namilumab, Otilimab, Gimsilumab, and TJM2 (TJ003234). In some embodiments, the hypomethylating agent is selected from the group consisting of azacytidine, decitabine, and a combination of decitabine and cedazuridine (INQOVI®). In a particular embodiment, the hypomethylating agent is azacitidine. In certain embodiments, the hypomethylating agent is administered for five to seven days starting on day one of administration of the anti-hGM-CSF antibody. In some embodiments of said methods, the anti-hGM-CSF antibody is lenzilumab. In certain embodiments of said methods, the anti-hGM-CSF antibody is selected from the group consisting of Namilumab, Otilimab, Gimsilumab, and TJM2 (TJ003234). In a particular embodiment of the herein provided methods, the anti-hGM-CSF antibody lenzilumab is administered on day one and day 15 of cycle 1 and on day one only for all subsequent cycles. In an embodiment, the all subsequent cycles consist of a total of 24 cycles, and each cycle consists of 28 days. In some embodiments, the anti-hGM-CSF antibody clected from the group consisting of Namilumab, Otilimab, Gimsilumab, and TJM2 (TJ003234) is administered on day one and day 15 of cycle 1 and on day one only for all subsequent cycles. In some embodiments, the all subsequent cycles consist of a total of 24 cycles, and cach cycle consists of 28 days. In particular embodiments, the subject has a RAS pathway mutation in tumor cells of the subject, and a TET2 mutation. In certain embodiments, the subject has a RAS pathway mutation and two TET2 mutation variants in tumor cells of the subject. In a particular embodiment, the anti-hGM-CSF antibody lenzilumab is administered intravenously (IV) at a dosc of from 552 mg to 1656 mg, wherein the dose of 552 mg is administered over a 1 hour IV infusion and the dose of 1656 mg is administered over 2 hours IV infusion. In some embodiments, the anti-hGM-CSF antibody lenzilumab is administered at a dose of 552 mg over a 1 hour IV infusion. In an embodiment, the azacitidine, decitabine, or the combination of decitabine and cedazuridine is administered subcutaneously at a dose of 75 mg/m². In certain embodiments, the combination therapy, i.e., the administration of the anti-hGM-CSF antibody and the hypomethylating agent, demonstrates/achieves a complete response (CR) plus partial response (PR) of 50-90% compared to a CR+PR rate of 10-17% achieved with sole administration of a hypomethylating agent. In a particular embodiment, the anti-hGM-CSF antibody is lenzilumab. In some embodiments of said methods, the anti-hGM-CSF antibody is selected from the group consisting of Namilumab, Otilimab, Gimsilumab, and TJM2 (TJ003234). In an embodiment, the response to said combination therapy is a complete response or a partial response during 6 first cycles. In some embodiments, the CR or PR result in improved survival and progression-free survival at two years after treatment compared to survival and progression-free survival at two years after administration of a hypomethylating agent alone. In a particular embodiment, the administration demonstrates/achieves clinical benefit at any point during 24 cycles. In an embodiment, the clinical benefit comprises impact on physical and functional capacity of the subject; social well-being of the subject, hematological and non-hematologic safety and combinations thereof. In an embodiment of the provided methods, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In certain embodiments, the hematological safety comprises (a) an improved bone marrow response of less than 5% blasts within 12 months or (b) a complete response in subjects having a medium increase in GM-CSF and pro-inflammatory cytokines found in an innate immune response and MI macrophage activation compared to levels of GM-CSF and pro-inflammatory cytokines and MI macrophage activation in healthy subjects within 12 months. In some embodiments, the clinical benefit comprising impact on the physical capacity of the subject comprises a reduction in splenomegaly. In an embodiment, the hematological safety comprises a decrease in a variant allele frequency (VAF) of at least one identified RAS-pathway mutation, wherein the RAS-pathway mutation is KRAs and/or CBL. In certain embodiments, the provided methods further comprise treating the subject with an allogeneic transplant.

DNA sequencing is performed by whole genome sequencing or whole exome sequencing (targeted sequencing) techniques detect the RAS (NRAS, KRAS, CBL) and/or TET2 gene mutations. Both blood and bone marrow may be used for screening to detect bone marrow blasts. peripheral blood cell counts, and mutational status.

The CMML patients in Example I are relapsed refractory CMML patients.

### Lenzilumab

Lenzilumab (also referred to herein as “LENZ”) is a proprietary Humaneered® first-in-class monoclonal antibody with best-in-class specificity and affinity that neutralizes human GM-CSF (hGM-CSF) by preventing binding to and hence signaling through its receptor, as described in U.S. Pat. Nos. 8,168,183 and 9,017, 674, each of which is incorporated herein by reference in its entirety.

LENZ is a recombinant monoclonal antibody, derived from mouse antibody LMM102, targeting hGM-CSF, with potential immunomodulatory activity, high binding affinity in the picomolar range, 94% homology to human germline, and has low immunogenicity. Following intravenous administration, lenzilumab binds to and neutralizes GM-CSF, preventing hGM-CSF binding to its receptor, thereby preventing hGM-CSF-mediated signaling to myeloid progenitor cells.

Lenzilumab comprises a VH region (VH#5)
having the amino acid sequence:
(SEQ ID NO: 1)
QVQLVQSGAEVKKPGASVKVSCKASGYSFTNYYIHWVRQAPGQRLEWMGW
INAGNGNTKYSQKFQGRVTITRDTSASTAYMELSSLRSEDTAVYYCVRRQ
RFPYYFDYWGQGTLVTVSS
and
a VL region (VK#2) having the amino
acid sequence:
(SEQ ID NO: 2)
EIVLTQSPATLSVSPGERATLSCRASQSVGTNVAWYQQKPGQAPRVLIYS
TSSRATGITDRFSGSGSGTDFTLTISRLEPEDFAVYYCQQFNKSPLTFGG
GTKVEIK

### Azacitidine and Other Demethylating Agents

A hypomethylating agent (or demethyating agent) is a drug that inhibits DNA methylation, i.e., the modification of DNA nucleotides by addition of a methyl group. Azacitidine is an analog of the nucleoside cytidine that inhibits DNA methyltransferase, impairing DNA methylation. Azacitidine (also referred to herein as “AZA”) is a ribonucleoside, and thus incorporates primarily into RNA to a larger extent than into DNA. Azacitidine's antineoplastic activity comprises two mechanisms: (1) the inhibition of DNA methyltransferase at low doses, causing hypomethylation of DNA, and (2) direct cytotoxicity in abnormal hematopoietic cells in the bone marrow through its incorporation into DNA and RNA at high doses, resulting in cell death. Oral azacitidine (ONUREG) is used for continued treatment of adult patients with acute myeloid leukemia (AML), which achieved first complete remission (CR) or complete remission with incomplete blood count recovery (CRi) following intensive induction chemotherapy and are not able to complete intensive curative therapy.

The hypomethylating agent decitabine (5-aza-2′-deoxycytidine) is a deoxyribonucleoside that only incorporates into DNA. Decitabine is used to treat blood/bone marrow disorders (myelodysplastic syndromes; “MDS”). Decitabine/cedazuridine (INQOVI®) is a combination of decitabine and cedazuridine, a cytidine deaminase inhibitor. Decitabine/cedazuridine is used to treat adults with myelodysplastic syndromes (MDS), including CMML.

### Pharmaceutical Compositions

The anti-hGM-CSF antibodies and the hypomethylating agents described and administered according to the methods provided herein are formulated as pharmaceutical compositions comprising an anti-hGM-CSF antibody and a hypomethylating agent, respectively, and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or more therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, intra-vaginally, or intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl-and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL.TM. (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (anti-hGM-CSF antibodies and the hypomethylating agents, respectively) in the required amount to produce a therapeutic effect in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The pharmaceutical compositions and formulations comprising an anti-hGM-CSF antibody and the pharmaceutical compositions and formulations comprising a hypomethylating agent, as respectively described herein, may be administered alone or with other biologically-active agents. Administration can be systemic or local, e.g., through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopcia or other generally recognized pharmacopcia for use in animals and, more particularly, in humans.

### Effective Doses

Effective doses of the pharmaceutical compositions of the present invention, for treatment of conditions or diseases, such as CMML, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.c., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, in particular CMML, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and cach patient's circumstances. In one embodiment, the pharmaceutical composition comprising an anti-hGM-CSF antibody is administered intravenously at a dose of from 552 mg to 1656 mg, wherein the dose of 552 mg is administered over a 1 hour IV infusion. In some embodiments, the pharmaceutical composition comprising an anti-hGM-CSF antibody is administered intravenously at a dose of 1656 mg over 2 hour(s) IV infusion. In a particular embodiment, the pharmaceutical composition comprises the anti-hGM-CSF antibody lenzilumab. In some embodiments, the pharmaceutical composition comprises an anti-hGM-CSF antibody selected from the group consisting of Namilumab, Otilimab, Gimsilumab, and TJM2 (TJ003234).

The hypomethylating agent is administered subcutaneously at a dose of 75 mg/m2. In certain embodiments of the pharmaceutical composition comprising a hypomethylating agent, the hypomethylating agent is selected from the group consisting of azacytidine, decitabine, and a combination of decitabine and cedazuridine.

The compound(s) or composition(s) of the invention may be administered only once, or it may be administered multiple times. For multiple dosages, the composition may be, for example, administered three times a day, twice a day, once a day, once every two days, twice a week, weekly, once every two weeks, or monthly.

The following examples are presented in order to more fully illustrate the preferred embodiments of the invention. They should in no way be construed, however, as limiting the broad scope of the invention.

### EXAMPLE 1

### Treatment of CMML with Anti-GM-CSF Antibody Lenzilumab and Azacitidine

The PREcision Approach to CHronic Myelomonocytic Leukemia (PREACH-M) trial assesses the efficacy of LENZ in CMML (ACTRN12621000223831p) to improve outcomes beyond those afforded by SOC.

### Methods

PREACH-M is a Phase 2/3 non-randomized, open-label precision medicine trial in 72 adults aged at least 18 years, newly diagnosed with WHO 2016 criteria for CMML; cytopenia (hemoglobin <100 g/L, platelets <100×10⁹/L or absolute neutrophil count <1.8×10⁹/L): white cell count ≥13×109/L; as well as TET2 (tet methylcytosine dioxygenase 2) and/or RAS pathway mutations (NRAS, KRAS, CBL).

Key exclusion criteria include prior treatment with investigational agents; radiotherapy within 28 days before treatment; treatment with G-CSF within 7 days of screening; GM-CSF within 28 days of screening; and uncontrolled medical conditions.

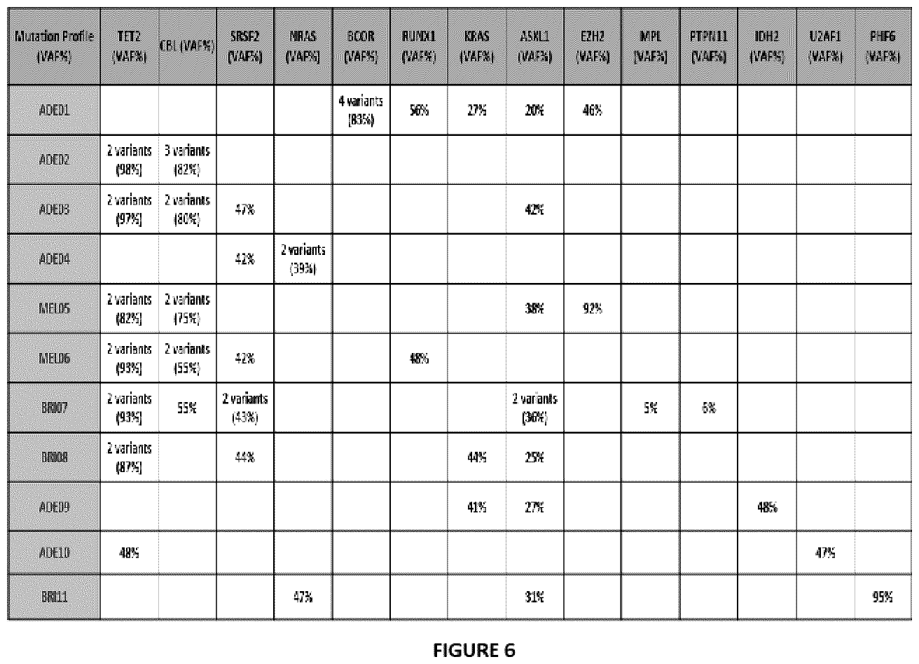

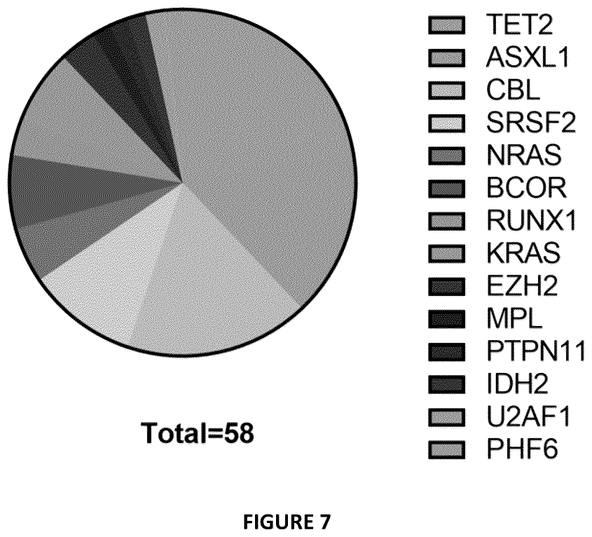

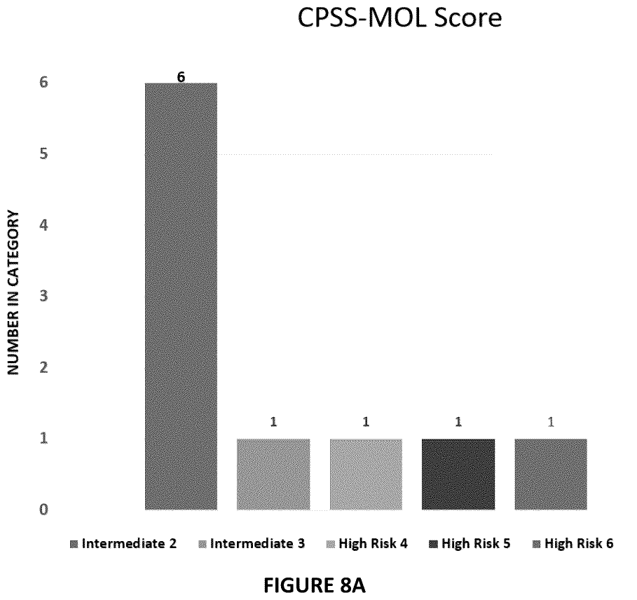

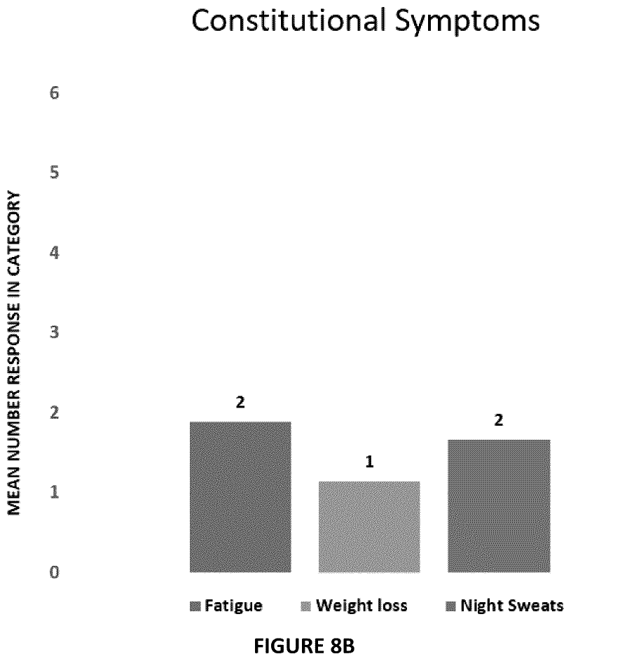

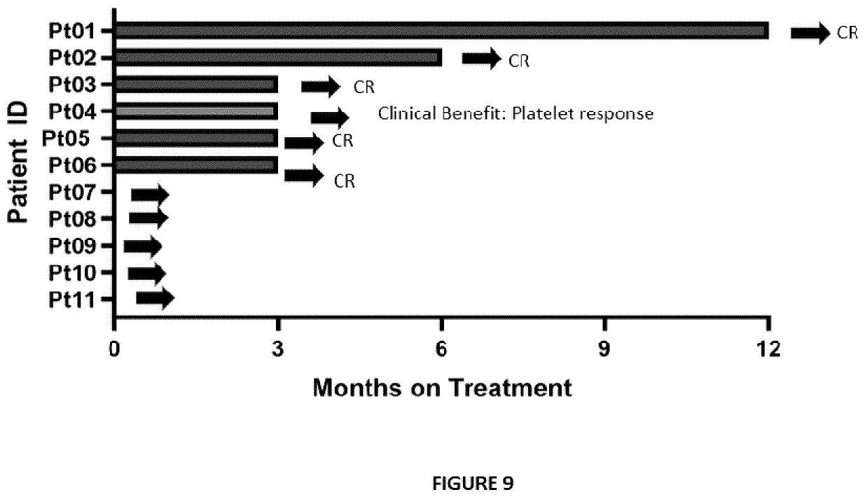

Subjects exhibiting RAS pathway mutations in their tumor cells, with or without TET2 mutations, receive 24 cycles (28 days) of azacitidine (SC; 75 mg/m² for 7 days) and LENZ (IV; 552 mg; d1 & d15 of cycle 1 and d1 only for all subsequent cycles); while those with non-RAS pathway mutations in their tumor cells, receive the same azacitidine regimen and sodium ascorbate (IV; 30 g for 7 days [15 g for 1st dose only, 30 g thereafter if no evidence of tumor lysis syndrome]; PO; 1.1g on all other days). FIG. 6 shows the gene mutation profile baseline characteristics of 11 subjects in the lenzilumab cohort, including NRAS, KRAS, and/or CBL mutations and TET2 mutations. Fig.7 shows in a pie chart format the genes mutated in the lenzilumab cohort. Cbl mutations are frequent, as are TET2 mutations.

Subjects who complete 24 cycles of treatment are followed every 6 months for 24 months for survival, disease status, and CMML-related therapy.

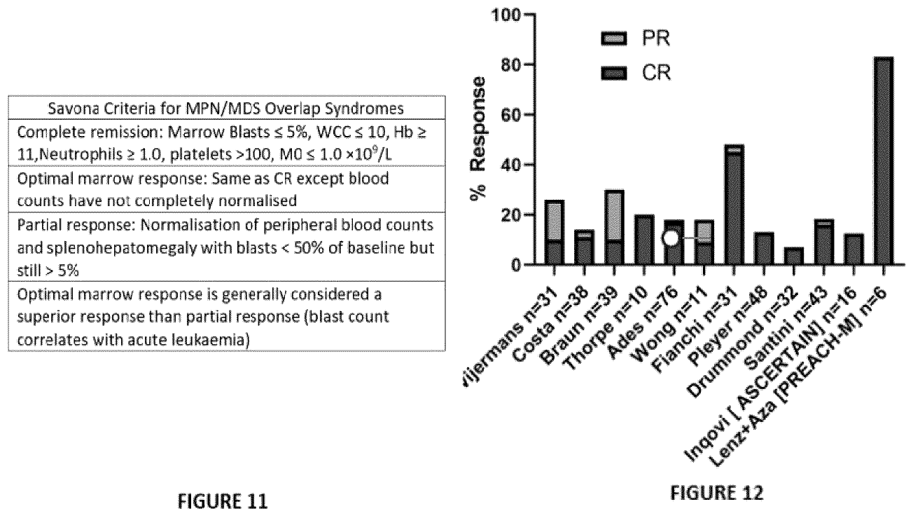

The primary endpoint is the frequency of CR or PR at any time during the first 12 cycles according to Savona Criteria (FIG. 11). Secondary endpoints include overall survival and progression-free survival at 2 years; proportion of subjects with clinical benefit at any point during the 24 cycles; impact on physical and functional capacity; social well-being according to Multidimensional Geriatric Assessment and quality of life; as well as hematological and non-hematologic safety.

### Results

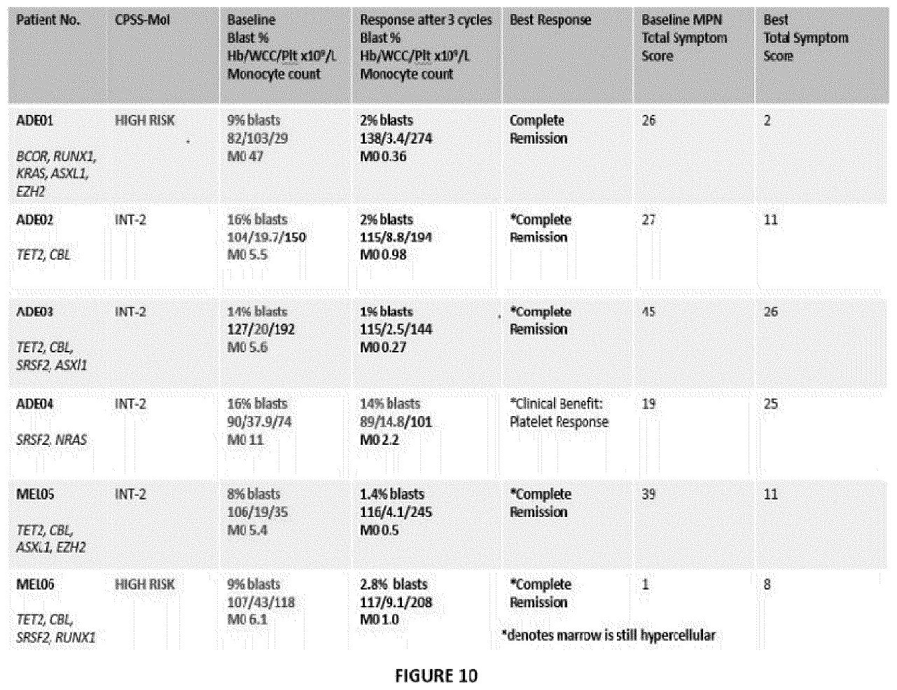

As of Dec. 31, 2022, eleven subjects were treated with azacitidine and LENZ (5 females, mean age of 67 years; 3 males, mean age of 69 years); among them 6 were evaluable based on at least 3 months of follow-up. Complete response (CR) or objective responses were observed in all evaluable patients including 2 with high risk based on molecular profiling (FIG. 10).

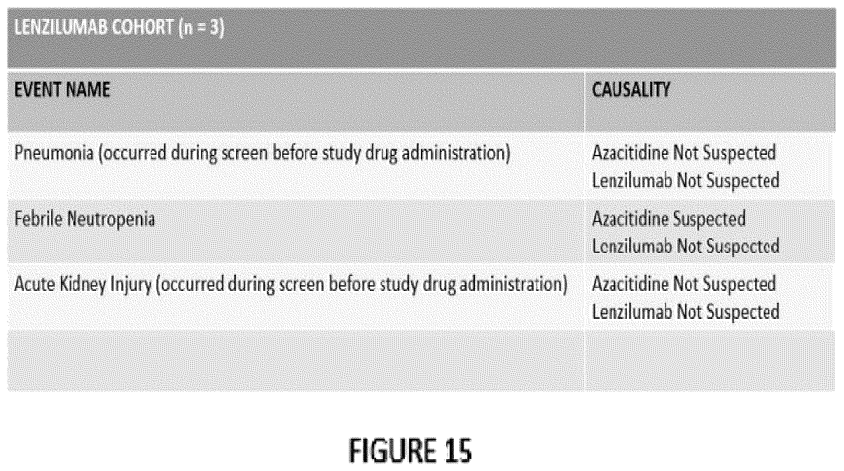

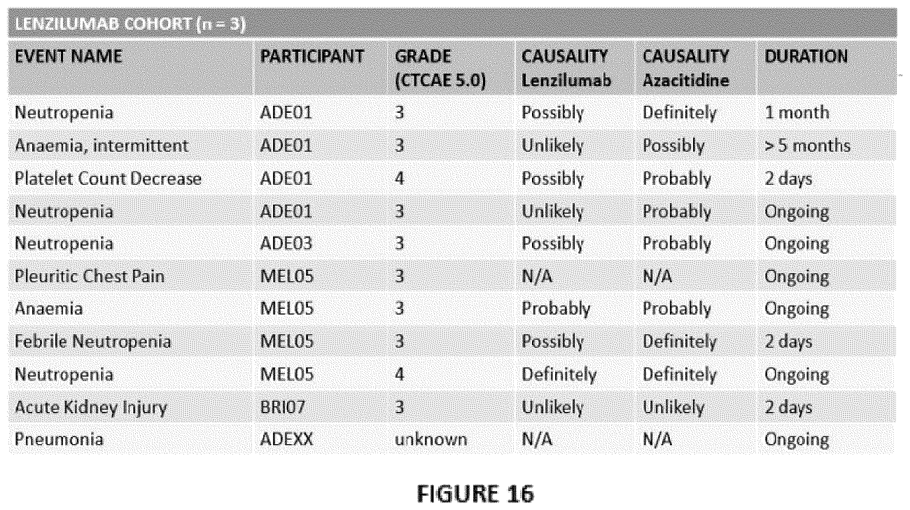

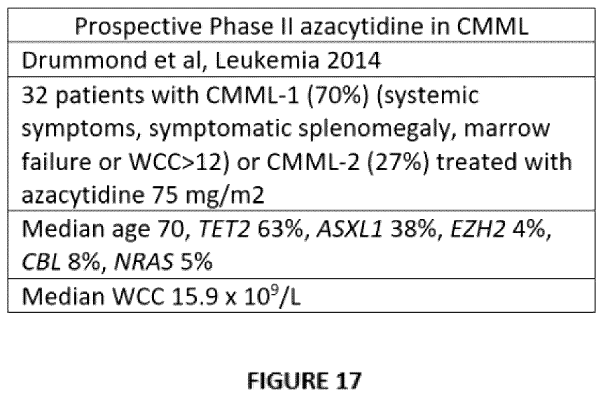

10 grade 3/4 Serious Adverse Events (AE) were observed of which 2 were assessed by the investigator as possibly related to LENZ. (FIGS. 15 and 16) Regarding the adverse effects summarized in FIG. 16, the investigators made a judgement regarding whether, in their opinion, the adverse event was related to a study drug. Options will include:

Definitely: The investigator feels that there is compelling evidence that there is a direct causal relationship between the AE and administration of the study drug.

Probably: there is evidence to suggest a causal relationship, and the influence of other causalities is unlikely.

Possibly: there is some evidence to suggest a causal relationship (i.e., there is a reasonable possibility that the AE may have been caused by the study drug). However, the role of other factors may have contributed to the event or it is possible that other factors may have been responsible for the event.

Unlikely: there is little evidence to suggest that there is a causal relationship with a study drug and there is another reasonable explanation for the event.

Not applicable: the participant did not receive study drug.

AZA is known to cause neutropenia, but lenzilumab has never been associated with neutropenia in any other studies. This is the first time that anti-hGM-CSF antibody, LENZ, and AZA have been used together in human subjects. Although LENZ does not cause neutropenia when dosed alone it is possible that AZA+LENZ could cause neutropenia.

### Conclusion

The ongoing PREACH-M trial evaluated GM-CSF neutralization with LENZ in addition to standard of care (AZA), in the treatment of patients having CMML with RAS pathway mutations or RAS and TET2 mutations in their tumor cells.

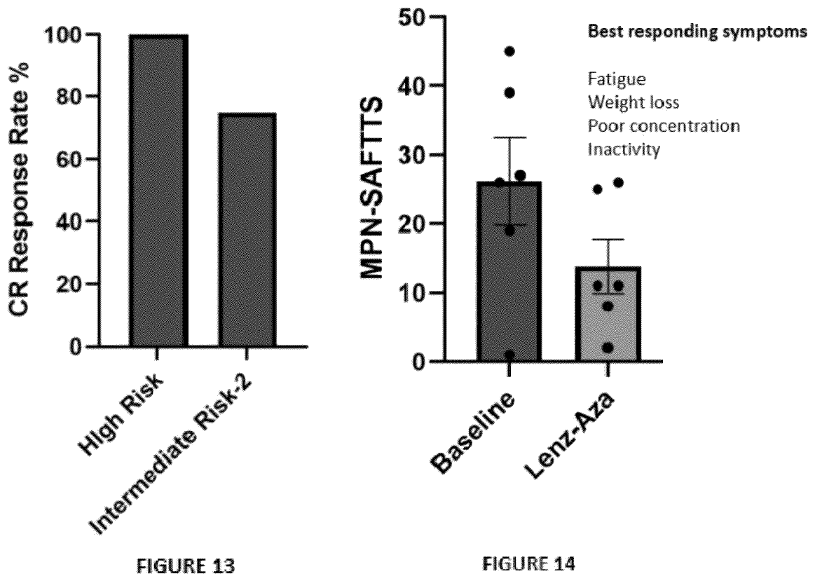

A superior carly response data of Lenz+Aza combination therapy for CMML was shown compared to published literature for CMML treatment with DNA methyltransferase inhibitors (DNMTi) alone. (FIG. 12) A high CR response rate (%) obtained from Lenz +Aza combination therapy in high risk CMML patients was also shown. (FIG. 13). Likewise, a robust improvement in quality of life based on the MPN symptoms assessment form: total symptom score compared to baseline scores was demonstrated, of which the best responding symptoms were fatigue, weight loss, poor concentration, and inactivity.

Having described certain embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

### EXAMPLE 2

Lenzilumab in Addition to Azacitidine Improves Complete Response Rates in Chronic Myelomonocytic Leukemia

Chronic myelomonocytic leukemia (CMML) is a rare cancer orchestrated by granulocyte-macrophage colony-stimulating factor (GM-CSF), a pro-inflammatory cytokine that drives leukemic monocyte proliferation. Standard of care (SOC) for CMML treatment includes azacitidine (AZA), with a complete response (CR) rate of 16-21% (as described by Xu Y, et al., Real-world data on efficacy and safety of azacitidine therapy in chronic myelomonocytic leukemia in China: results from a multicenter, retrospective study. Invest New Drugs 2022;40(5): 1117-1124. DOI:10.1007/s10637-022-01283-x, and Zheng X, et al., Efficacy and Safety of Hypomethylating Agents in Chronic Myelomonocytic Leukemia: A Single-Arm Meta-analysis. Glob Med Genet 2022;9(2): 141-151. DOI: 10.1055/s-0042-1744157, each of which is incorporated herein by reference in its entirety).

The PREcision Approach to Chronic Myelomonocytic Leukemia (PREACH-M; ACTRN12621000223831) trial investigates novel CMML therapies directed by molecular profiling. Lenzilumab (LENZ; Humanigen, Inc., Short Hills, NJ) a proprietary Humaneered® first-in-class monoclonal antibody with best-in-class off-rate and affinity that neutralizes GM-CSF. PREACH-M interim results show that LENZ/AZA improves hematologic parameters, decreases spleen size, and dampens pro-inflammatory responses in CMML with RAS-pathway mutations. This report details the objective clinical responses from an interim analysis of the first 11 subjects who completed at least three months LENZ/AZA treatment.

### Methods

PREACH-M is a Phase 2/3 nonrandomized, uncontrolled, open-label trial in 72 adults aged at least 18 years, newly diagnosed with WHO 2016 criteria for CMML. Key exclusion criteria include prior treatment with investigational agents; radiotherapy within 28 days before treatment; treatment with G-CSF within 7 days of screening; GM-CSF within 28 days of screening; and uncontrolled medical conditions. Subjects exhibiting RAS pathway mutations (NRAS, KRAS, CBL) receive 24 cycles (every 28 days) of AZA (SC; 75 mg/m2 for 7 days) and LENZ (IV; 552 mg; dl & d15 of cycle 1 and dl only for all subsequent cycles); while those with only TET2 mutations receive the same AZA regimen and sodium ascorbate (IV; 30 g for 7 days [15 g for 1st dose only, 30 g thereafter if no evidence of tumor lysis syndrome]; PO; 1.1g on all other days). Subjects who complete 24 cycles of treatment are followed every 6 months for an additional 24 months. The primary endpoint is the frequency of complete response (CR) or partial response (PR) during the first 12 cycles according to Savona Criteria. Secondary endpoints include responses according to modified 2006 International Working Group criteria, 2 year overall survival, and symptom improvement.

### Results

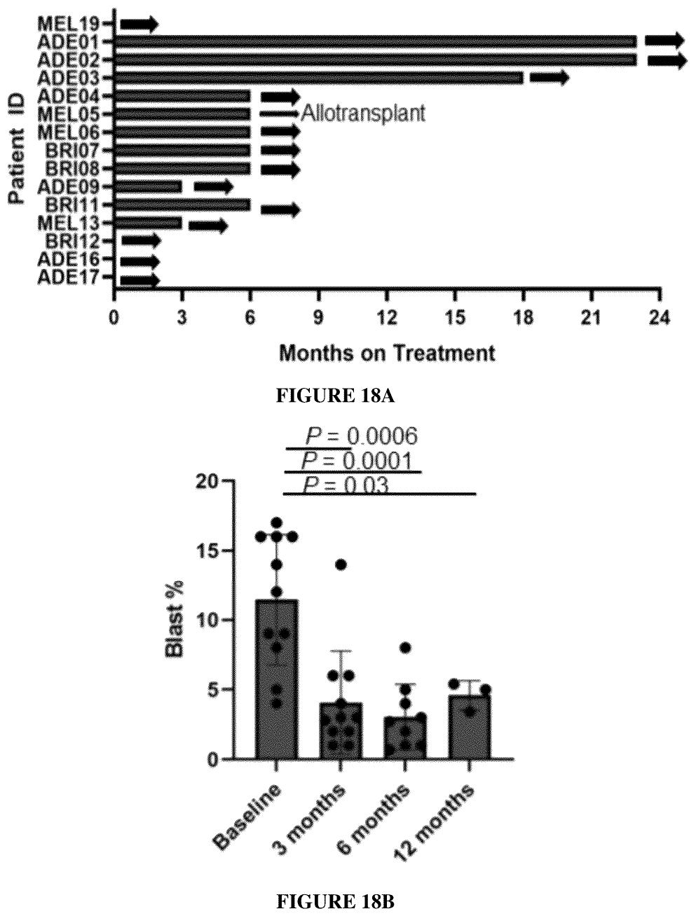

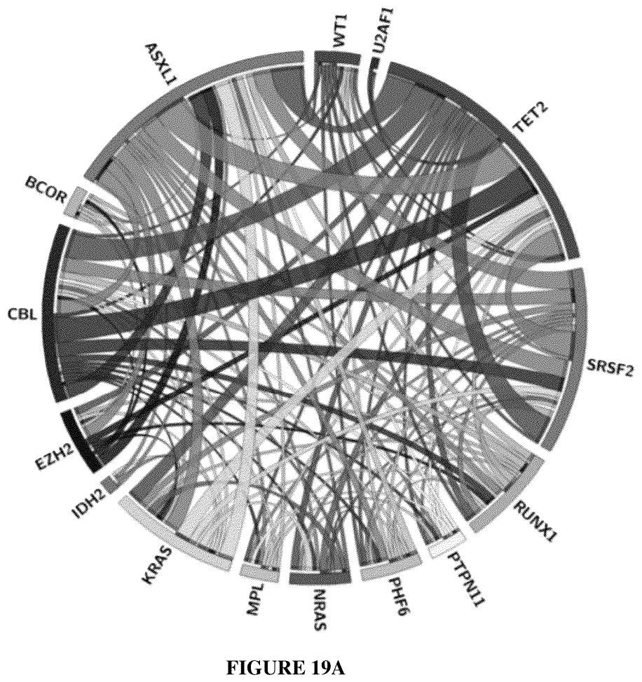

As of July 2023, 15 subjects were enrolled in the LENZ/AZA arm (8 females, 7 males with mean age 69; mean white cell count 21×10⁹/L, mean Hb 121 g/L; mean platelet count, 74×10⁹/L, mean blast count, 10.1%). (FIGS. 18A-18B) Mutations included; CBL (47% of subjects), NRAS (27%), KRAS (47%), NRAS and KRAS (13%), and TET2 (93%). Subjects exhibited CPSS-MOL scores, of intermediate risk 1 (n=1), intermediate risk 2-3 (n=8), and high risk 4-6 (n=6). All the 11 evaluable subjects at 3 months responded to LENZ/AZA. (FIG. 18A) CR was achieved within 3 cycles in 55% of subjects. Six of the subjects demonstrated CR including 2 with a high risk CPSS-MOL profile and 8 achieved either CR or complete marrow response (blasts<5%) within 12 months. (FIG. 18B) One subject had a platelet response, 1 subject cach had PR and stable disease with blasts <5%. CMML progression was absent and I subject became eligible for allogencic transplant. These findings exceed historical CR rates for hypomethylating agents (16%; 95% CI, 12-21% (as described by Xu Y, et al., 2022 supra) and 21%; 13-29% (as described by Zheng X, et al., 2022 supra)). Self-reported symptom scores from the standardized MPN-SAFFS improved from baseline (mean of 22 vs 12, P=0.06). Fifteen grade 3 and 9 grade 4 adverse events were reported of which 2 were “probably” ascribed to both LENZ/AZA and 7 were “possibly” ascribed to LENZ. No unexpected adverse events were observed.

### Conclusion

Interim analysis of the PREACH-M trial demonstrated that GM-CSF neutralization with LENZ/AZA, for the treatment of CMML with RAS-pathway mutations resulted in 55% CR, achieved early in treatment, durability up to 18 months, thus far, and no unexpected serious adverse events. These data suggest CMML is driven by a non-redundant cytokine that responds to immunotherapy.

### EXAMPLE 3

### Suppression of KRAS and CBL Mutations and Hematological Improvement by Lenzilumab and Azacitidine Treatment in Proliferative Chronic Myelomonocytic Leukemia

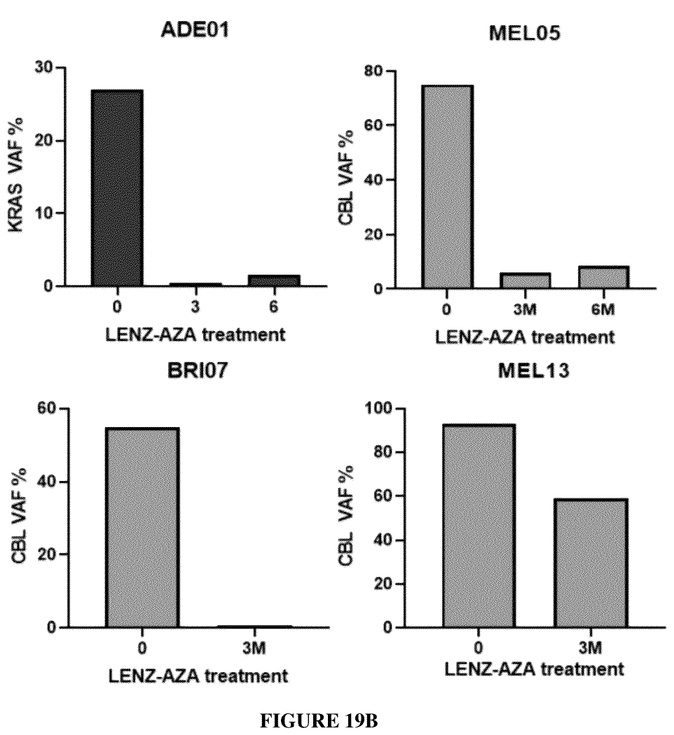

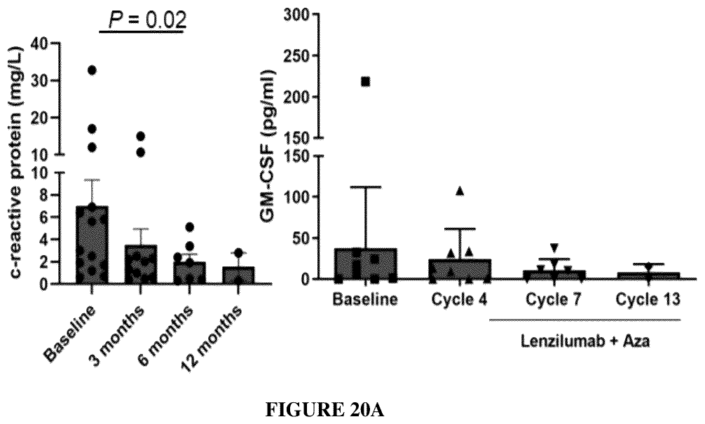

Mutations in RAS-pathway genes (NRAS, KRAS, CBL) are amongst the most common somatic mutations in cancer and historically resistant to most therapies. (FIG. 19A) Targeted therapies that impact the RAS-pathway constitute a major unmet need in oncology. The proliferative form of chronic myelomonocytic leukemia (CMML) is commonly associated with RAS-pathway mutations and has a high propensity to develop into acute myeloid leukemia. In pre-clinical models, Lenzilumab (LENZ; Humanigen, Inc., Short Hills, NJ), a proprietary Humancered® first-in-class monoclonal antibody with best-in-class off-rate and affinity that neutralizes GM-CSF, resulted in a reduction of colony numbers and viability of CMML cells, with the greatest sensitivity in cells possessing RAS-pathway (NRAS/KRAS/CBL) mutations, suggesting benefit in the targeted treatment of CMML. (FIG. 19B) The PREcision Approach to CHronic Myelomonocytic Leukemia (PREACH-M) trial assesses the efficacy of LENZ, in addition to azacytidine (AZA), in CMML subjects with RAS-pathway mutations and high dose sodium ascorbate (ASC) and AZA in CMML subjects without RAS-pathway mutations. Interim data from 11 subjects with RAS-pathway (NRAS/KRAS/CBL) mutations in PREACH-M receiving LENZ/AZA demonstrate reductions in circulating GM-CSF and CRP with 8 subjects achieving complete response or optimal marrow response. This Example describes improvements in variant allele frequencies (VAF) for RAS-pathway mutations (FIG. 19B) and hematologic improvements associated with LENZ/AZA treatment in CMML.

### Methods

PREACH-M is a Phase 2/3 non-randomized, open-label trial in 72 subjects, aged at least 18 years, with newly diagnosed CMML based on the WHO 2016 criteria; and RAS-pathway mutations at a variant allele frequency >3%. Subjects received 24 cycles (every 28 days) of AZA (SC; 75 mg/m2 for 7 days) and LENZ (IV; 552 mg; dl & d15 of cycle 1 and dl only for all subsequent cycles). Subjects without RAS-pathway mutations received the same AZA regimen and sodium ascorbate (IV; 30g for 7 days (15g for 1st dose only; 30g thereafter if no evidence of tumor lysis syndrome); PO; 1.1g on all other days). VAF was determined from bone marrow mononuclear cells using a 41-myeloid panel using Illumina Hi-Seq with a depth of 1000x performed on bone marrow aspirates obtained at baseline and Day 1 of treatment cycles 4, 7, and 12.

### Results

As of July 2023, 11 subjects completed at least 3 months of LENZ/AZA treatments and follow-up sequencing data were available for 10 subjects. Five of 10 subjects showed >10% decrease in VAF for at least one mutation detected at baseline, including 5 decreased alleles in the RAS-pathway (KRAS, CBL) out of 22 total VAF mutation responses. (FIG. 19B) CBL mutations showed the largest change with a decrease in >50% VAF in three subjects. (FIG. 19B) Decreases in RAS-pathway clones by LENZ/AZA were durable at 6 months. No decreases in VAF for any mutations were observed in 2 evaluable subjects following ASC/AZA treatment. Following 3 months LENZ/AZA treatment, blood monocyte count improved from a baseline of 11.0±6.0×10⁹/L to 2.0±1.0×10⁹/L (p=0.030). Blast differential improved from 10.5±5.0% to 4.0±3.5% (p=0.038). Platelet count increased from 90.0±50.0×10⁹/L to 150.0±60.0×10⁹/L (p=0.010). Statistical improvements remained durable for at least 6 (n=6) and 12 (n=3) months. Hemoglobin concentration increased from 100.0±22.0 g/L to 115.0±10.0 g/L at 3 months (p=NS, n=11) and progressively increased to 122.0±10.0 g/L at 12 months (p=0.024, n=3). Spleen length decreased from 15.0±4.0 cm to 13.0±4.0 cm (p=0.03) and decreased further to 11.0±2.0 cm in subjects who received 12 months of treatment (n=3). No evidence of disease progression or relapse was observed in any subject.

### Conclusion

In 11 evaluable subjects with proliferative CMML and RAS-pathway mutations, GM-CSF neutralization with LENZ in addition to AZA standard of care, resulted in significant decreases in the proportion of KRAS and CBL mutant leukemic cells, accompanied by clinically significant hematologic improvements and a reduction in splenomegaly. Lenzilumab may have efficacy in preventing outgrowth of RAS-pathway mutations, specifically KRAS and CBL, in the context of CMML and other myeloid malignancies.

### EXAMPLE 4

### Cytokine and Mutation Profiling Reveal Patterns of Complete Remission Rates with Lenzilumab Combination Therapy in Chronic Myelomonocytic Leukemia

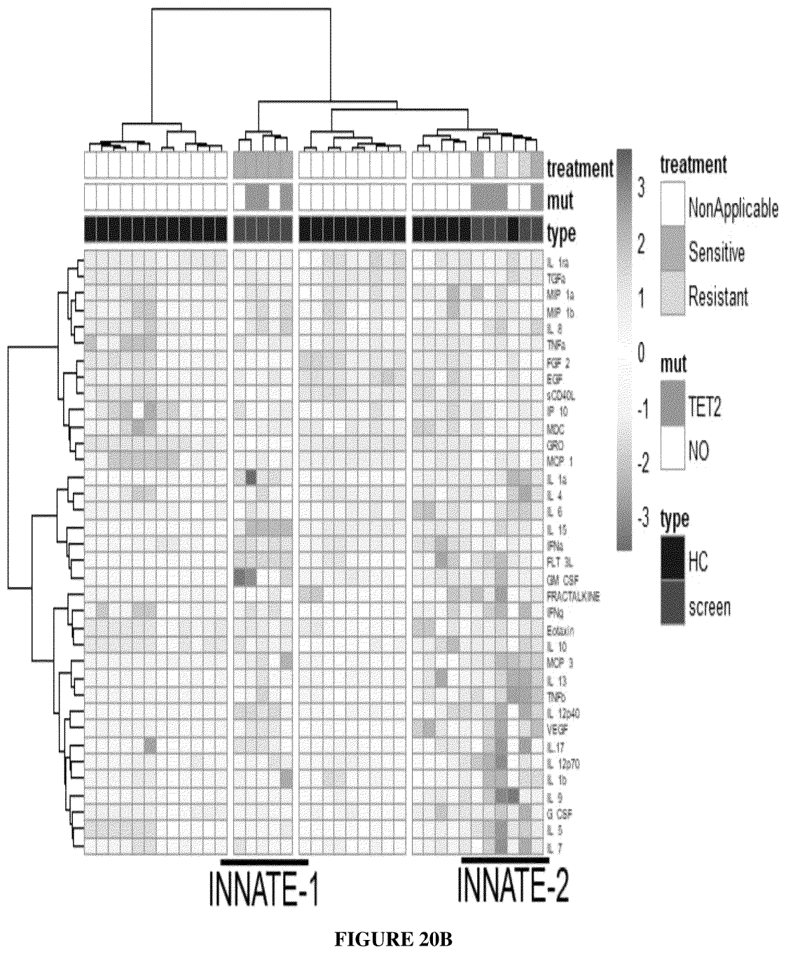

Chronic myelomonocytic leukemia (CMML) is characterized by accumulation of classical CD14+CD16-inflammatory monocytes driven in part by hypersensitivity to granulocyte-macrophage colony-stimulating factor (GM-CSF), a pro-inflammatory cytokine. Standard of care in CMML includes hypomethylating agents such as azacytidine (AZA), with complete response (CR) rates of 16-21% (as described by Xu Y., et al. 2022 supra and Zheng X ., et al., 2022 supra) and no reliable biomarkers that predict response. The complete pro-inflammatory profile of CMML is unknown and no treatment addresses the hematologic aberrations of CMML. Lenzilumab (LENZ; Humanigen, Inc., Short Hills, NJ) is a proprietary Humancered® first-in-class monoclonal antibody with best-in-class off-rate and affinity that neutralizes GM-CSF. The interim analysis of the PREcision Approach to CHronic Myelomonocytic Leukemia (PREACH-M; ACTRN12621000223831) trial showed LENZ/AZA treatment in 11 subjects with RAS-pathway (NRAS/KRAS/CBL) mutations resulted in 8 subjects with complete response or optimal marrow response and improvements in hematological parameters. This Example describes the cytokine profiles and systemic C-reactive protein levels of these subjects. (FIG. 20A)

### Methods

PREACH-M is a Phase 2/3 non-randomized, open-label trial in 72 subjects, aged at least 18 years, with newly diagnosed CMML based on the WHO 2016 criteria; and RAS-pathway mutations at a variant allele frequency >3%. Subjects received 24 cycles (every 28 days) of AZA (SC; 75 mg/m2 for 7 days) and LENZ (IV; 552 mg; dl & d15 of cycle 1 and dl only for all subsequent cycles). Subjects without RAS-pathway mutations received the same AZA regimen and sodium ascorbate (IV; 30 g for 7 days (15 g for 1st dose only; 30 g thereafter if no evidence of tumor lysis syndrome); PO; 1.1 g on all other days). Cytokine profiling from bone marrow plasma was performed after 4, 7, 12 and 24 months using Milliplex Human Cytokine/Chemokine Magnetic bead panel and compared with 24 age-matched healthy subjects. Unsupervised hierarchical clustering with Ward's method sought distinct CMML patterns based on cytokine expression. C-reactive protein (CRP) was determined from blood samples using a routine assay. Variant allele frequencies were determined from bone marrow mononuclear cells using a 41-myeloid panel using Illumina Hi-Seq with a depth of 1000x.

### Results

As of Jul. 2023, 15 subjects were enrolled in the LENZ/AZA arm (8 females, 7 males with mean age 69; mean white cell count, 21x109/L; mean Hb, 121 g/L, mean platelet count; 74×10⁹/L, mean blast count, 10.1%). CRP decreased from a median of 5.6 at baseline to 2.1 mg/L after 6 months of LENZ/AZA (P=0.02). (FIG. 20A)

GM-CSF and other pro-inflammatory cytokines were increased in CMML bone marrow plasma compared with healthy subjects. Hierarchical clustering of all subjects revealed 2 patterns of CMML, distinct from healthy subjects. Cluster “INNATE-1” was comprised of medium increases in inflammatory cytokines associated with innate immune response and MI macrophage activation (IFN-g, IL-1b, TNFa, IL-12p70, IL-12p40, IL-17, Fractalkine, MCP-3). Cluster “INNATE-2” was associated with extreme increases of these cytokines. The mean proinflammatory innate immune score (sum of z-scores) was 20-fold greater in INNATE-2 compared with INNATE-1 (P=0.055). Somatic mutations SRSF2, WTI, PHF6 were enriched in INNATE-2. All patients (5/5) in INNATE-1 showed 100% response (CR or optimal marrow response resulting in <5% blasts) to AZA/LENZ. (FIG. 20B) Two subjects (2/6) in INNATE-2 demonstrated partial responses or hematological improvement thus far.

### Conclusion

CMML is a disorder of profound innate immune activation, driven by GM-CSF and other pro-inflammatory cytokines. Early treatment with LENZ/AZA, a precision immunotherapeutic approach, leads to a) efficacy in INNATE-1 (FIG. 20B) that exceeds historical CR rates for hypomethylating agents (as described by Xu Y., et al. 2022 supra and Zheng X ., et al., 2022 supra); and b) evolving efficacy in INNATE-2, in which pro-inflammatory activity is more robust.

What is claimed is:

1. A method for treating a subject having chronic myelomonocytic leukemia (CMML), the method comprising:
a) identifying a RAS pathway mutation in tumor cells of the subject, wherein the RAS pathway mutation is a NRAS, KRAS, and/or CBL mutation; and
b) administering to the subject identified in step (a) a therapeutically effective amount of an anti-hGM-CSF antibody.

2. The method of claim 1, further comprising administering a therapeutically effective amount of a hypomethylating agent for five to seven days starting on day one of administration of the anti-hGM-CSF antibody.

3. The method of claim 2, wherein the hypomethylating agent is selected from the group consisting of azacytidine, decitabine, and a combination of decitabine and cedazuridine.

4. The method of claim 1, wherein the anti-hGM-CSF antibody is lenzilumab.

5. The method of claim 1, wherein the anti-hGM-CSF antibody is selected from the group consisting of Namilumab, Otilimab, Gimsilumab, and TJM2 (TJ003234).

6. The method of claim 2, wherein the anti-hGM-CSF antibody is lenzilumab.

7. The method of claim 2, wherein the anti-hGM-CSF antibody is selected from the group consisting of consisting of Namilumab, Otilimab, Gimsilumab, and TJM2 (TJ003234).

8. The method of claim 4, wherein the anti-hGM-CSF antibody lenzilumab is administered on day one and day 15 of cycle 1 and on day one only for all subsequent cycles.

9. The method of claim 8, wherein the all subsequent cycles consist of a total of 24 cycles, and each cycle consists of 28 days.

10. The method of claim 1, wherein the subject has a RAS pathway mutation and a TET2 mutation identified in the tumor cells.

11. The method of claim 1, wherein the subject has a RAS pathway mutation and two TET2 mutation variants identified in the tumor cells.

12. The method of claim 4, wherein the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hour(s) IV infusion.

13. The method of claim 4, wherein the anti-hGM-CSF antibody lenzilumab is administered at a dose of 552 mg over a 1 hour IV infusion.

14. The method of claim 3, wherein the hypomethylating agent is administered subcutaneously at a dose of 75 mg/m².

15. The method of claim 2, wherein the administration demonstrates a complete response (CR) plus partial response (PR) of 50-90% compared to complete response (CR) plus partial response (PR) of 10-17% achieved with sole administration of a hypomethylating agent.

16. The method of claim 15, wherein the response is a complete response or a partial response during 6 first cycles.

17. The method of claim 15, wherein the CR or PR result in improved survival and progression-free survival at two years after treatment compared to survival and progression-free survival at two years after administration of a hypomethylating agent alone.

18. The method of claim 2, wherein the administration demonstrates/achieves clinical benefit at any point during 24 cycles.

19. The method of claim 18, wherein the clinical benefit comprises impact on physical and functional capacity of the subject; social well-being of the subject, hematological and non-hematologic safety and combinations thereof.

20. A method for treating a subject having chronic myelomonocytic leukemia (CMML), the method comprising:
a) identifying a RAS pathway mutation in tumor cells of the subject, wherein the RAS pathway mutation is a NRAS, KRAS, and/or CBL mutation; and
b) administering to the subject identified in step (a) a therapeutically effective amount of an anti-hGM-CSF antibody and a therapeutically effective amount of a hypomethylating agent.

21. The method of claim 20, wherein the hypomethylating agent is selected from the group consisting of azacytidine, decitabine, and a combination of decitabine and cedazuridine.

22. The method of claim 20, wherein the hypomethylating agent is administered for five to seven days starting on day one of administration of the anti-hGM-CSF antibody.

23. The method of claim 20, wherein the anti-hGM-CSF antibody is lenzilumab.

24. The method of claim 20, wherein the anti-hGM-CSF antibody is selected from the group consisting of Namilumab, Otilimab, Gimsilumab, and TJM2 (TJ003234).

25. The method of claim 21, wherein the anti-hGM-CSF antibody is lenzilumab.

26. The method of claim 21, wherein the anti-hGM-CSF antibody is selected from the group consisting of Namilumab, Otilimab, Gimsilumab, and TJM2 (TJ003234)

27. The method of claim 23, wherein the anti-hGM-CSF antibody lenzilumab is administered on day one and day 15 of cycle 1 and on day one only for all subsequent cycles.

28. The method of claim 27, wherein the all subsequent cycles consist of a total of 24 cycles, and each cycle consists of 28 days.

29. The method of claim 20, wherein the subject has a RAS pathway mutation and a TET2 mutation identified in the tumor cells.

30. The method of claim 20, wherein the subject has a RAS pathway mutation and two TET2 mutation variants are identified in the tumor cells.

31. The method of claim 23, wherein the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hours IV infusion.

32. The method of claim 23, wherein the anti-hGM-CSF antibody lenzilumab is administered at a dose of 552 mg over a 1 hour IV infusion.

33. The method of claim 21, wherein the azacitidine, decitabine, or the combination of decitabine and cedazuridine is administered subcutaneously at a dose of 75 mg/m².

34. The method of claim 21, wherein administration of the anti-hGM-CSF antibody lenzilumab and the hypomethylating agent demonstrates a complete response (CR) plus partial response (PR) of 50-90% compared to a CR+PR rate of 10-17% achieved with sole administration of a hypomethylating agent.

35. The method of claim 34, wherein the response is a complete response or a partial response during 6 first cycles.

36. The method of claim 34, wherein the CR or PR result in improved survival and progression-free survival at two years after treatment compared to survival and progression-free survival at two years after administration of a hypomethylating agent alone.

37. The method of claim 21, wherein the administration demonstrates/achieves clinical benefit at any point during 24 cycles.

38. The method of claim 37, wherein the clinical benefit comprises impact on physical and functional capacity of the subject; social well-being of the subject, hematological and non-hematologic safety and combinations thereof.

39. The method of claim 19, wherein the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters.

40. The method of claim 19, wherein the hematological safety comprises (a) an improved bone marrow response of less than 5% blasts within 12 months or (b) a complete response in subjects having a medium increase in GM-CSF and pro-inflammatory cytokines found in an innate immune response and MI macrophage activation compared to levels of GM-CSF and pro-inflammatory cytokines and MI macrophage activation in healthy subjects within 12 months.

41. The method of claim 19, wherein the clinical benefit comprising impact on the physical capacity of the subject comprises a reduction in splenomegaly.

42. The method of claim 19, wherein the hematological safety comprises a decrease in a variant allele frequency (VAF) of at least one identified RAS-pathway mutation, wherein the RAS-pathway mutation is KRAs and/or CBL.

43. The method of claim 38, wherein the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters.

44. The method of claim 38, wherein the hematological safety comprises (a) an improved bone marrow response of less than 5% blasts within 12 months or (b) a complete response in subjects having a medium increase in GM-CSF and pro-inflammatory cytokines found in an innate immune response and Ml macrophage activation compared to levels of GM-CSF and pro-inflammatory cytokines and M1 macrophage activation in healthy subjects within 12 months.

45. The method of claim 38, wherein the clinical benefit comprising impact on the physical capacity of the subject comprises a reduction in splenomegaly.

46. The method of claim 38, wherein the hematological safety comprises a decrease in a variant allele frequency (VAF) of at least one identified RAS-pathway mutation, wherein the RAS-pathway mutation is KRAs and/or CBL.

47. The method of claim 2, further comprising treating the subject with an allogeneic transplant.

48. The method of claim 20, further comprising treating the subject with an allogeneic transplant.
